(19) United States
(12) Patent Application Publication
(10) Pub. No.: US 2025/0361486 A1
(43) Pub. Date: Nov. 27, 2025

(54) METHOD FOR CULTURING CELLS REQUIRING A SUPPLY OF IRON

(71) Applicant: SAFI BIOTHERAPEUTICS INC., Willmington, DE (US)

(72) Inventors: Guillaume ROUSSEAU, Paris (FR); Mathilde MAESTRALI, Paris (FR); Fanny RASSCHAERT, Paris (FR); Marie-Catherine GIARRATANA, Saint-Ouen (FR)

(21) Appl. No.: 18/872,618

(22) PCT Filed: Jun. 9, 2023
(86) PCT No.: PCT/EP2023/065558

§ 371 Date: Dec. 6, 2024

(30) Foreign Application Priority Data
Jun. 9, 2022 (FR) 2205547

(51) Int. Cl. C12N 5/078

(52) U.S. Cl. CPC C12N 5/0641; C12N 2500/24; C12N 2500/38

(57) ABSTRACT

The present invention relates to a method for culturing culture cells requiring a supply of iron, comprising a step of culturing cells to be cultured in a culture medium, the antioxidant capacity of which is higher than or equal to the antioxidant capacity of a solution of Trolox at a concentration of 10 μM.

### FIELD OF INVENTION

The present invention relates to a method for culturing cells requiring a supply of iron, and to a culture medium for culturing culture cells requiring a supply of iron.

### TECHNICAL BACKGROUND

It is known that, during storage, red blood cells intended for blood transfusion undergo hemolysis.

Among the solutions to solve this problem, Sparrow et al. (2014) Transfusion 54:560-568 proposed to replace the SAGM preservation solution (150 mM NaCl, 1.25 mM Adenine, 45 mM Glucose and 30 mM Mannitol) with the AS-1 preservation solution (154 mM NaCl, 1.25 mM Adenine, 111 mM Glucose and 41 mM Mannitol). Indeed, red blood cells stored in AS-1 solution exhibit significantly lower hemolysis from the 14^{th} day of storage compared to red blood cells stored in SAGM solution.

However, hemolysis still occurs in AS-1 solution, which it is important to be able to reduce.

### SUMMARY OF THE INVENTION

The present invention arises from the unexpected demonstration by the inventors that, in the context of a method for culturing cultured red blood cells, the addition to the culture medium of the antioxidant agent Trolox, at a concentration of 100 μM, made it possible to improve the conservation efficiency of the red blood cells after culture and to reduce cell losses at the end of culture.

The present invention relates to a method for culturing culture cells requiring a supply of iron, comprising a step of culturing cells to be cultured in a culture medium whose antioxidant capacity is greater than or equal to the antioxidant capacity of a 10 μM, in particular 50 μM, Trolox solution.

The present invention also relates to a culture medium intended for the culture of cells requiring a supply of iron, the antioxidant capacity of which is greater than or equal to the antioxidant capacity of a Trolox solution at 10 μM, in particular 50 μM, and is in particular less than the antioxidant capacity of a Trolox solution at 250 μM.

In a preferred embodiment of the culture method and the culture medium according to the invention, the culture medium comprises at least one antioxidant agent.

Advantageously, the culture medium according to the invention makes it possible to reduce cellular losses of cultured cells, in particular cultured red blood cells, at the end of culture, and/or to improve the conservation yield of cultured cells, in particular cultured red blood cells.

### DESCRIPTION OF THE INVENTION

As a preliminary point, it should be recalled that the term “comprising” means “including”, “containing” or “encompassing”, that is to say that when an object “comprises” one or more elements, elements other than those mentioned may also be included in the object. Conversely, the expression “consisting of” means “made up of”, that is to say that when an object “consists of” one element or several elements, the object cannot include other elements than those mentioned.

### Cultivation Process

The culture method according to the invention can be in batch mode, in fed-batch mode or by perfusion.

Perfusion is a continuous culture method in which cells are retained in the bioreactor or circulated and returned to the bioreactor while spent culture medium is removed, compensated by the addition of perfusion fluid to renew the culture medium. The used and evacuated culture medium therefore does not contain cells.

As used herein, a perfusion culture method comprises at least one step of culturing in a perfusion reactor.

Preferably, the culture method according to the invention is a perfusion culture method.

The culture step in a perfusion bioreactor according to the invention aims to multiply the cultured cells and, in the case of the production of cultured red blood cells, to complete their differentiation to bring them to a stage of reticulocyte, enucleated cell corresponding to a young or immature red blood cell, or up to a mature red blood cell stage.

The culture is carried out in a bioreactor adapted to perfusion culture. Many bioreactor models suitable for cell culture by perfusion are known to those skilled in the art.

The bioreactor preferably has a capacity of 0.5 to 5000 L. Preferably, the bioreactor has a capacity of at least 0.5, 1.2, 3, 4, 5, 6, 7, 8, 9, 10, 20, 30, 40, 50, 60, 70, 80, 90, 100, 200, 300, 400, 500, 600, 700, 800, 900, 1000, 2000, 3000 or 4000 L. Preferably, the bioreactor has a capacity of at most 5000, 4000, 3000, 2000, 1000, 900, 800, 700, 600, 500, 400, 300, 200, 100, 90, 80, 70, 60, 50, 40, 30, 20, 10, 9, 8, 7, 6, 5, 4, 3, 2 or 1 L.

Preferably, the bioreactor comprises a gas exchange means for satisfying the oxygen requirements of the cells and for controlling the pH by controlling the supply and/or removal of carbon dioxide (CO₂). Preferably, the gas exchange medium is low shear.

Preferably, at least one of the following culture conditions, more preferably all, are controlled or regulated:
Agitation;
The pH;
Dissolved oxygen (DO);
The temperature;
The volume or level of the bioreactor;
The perfusion rate;
The intake of nutrients, particularly chosen from carbohydrates, amino acids, vitamins and iron;
The supply of growth factors, cytokines and/or hormones;
Fouling of the bioreactor and clogging of the filtering organs.

Preferably, the culture is carried out for a period of time sufficient to obtain a cell concentration greater than 30 million cells/ml. Preferably this time period is 5 days to 25 days, more preferably 10 days to 20 days.

Preferably, the culture temperature is between 33° C. and 40° C., more preferably between 35° C. and 39° C., and even more preferably between 36° C. and 38° C.

Preferably, the culture pH is between 7 and 8, more preferably between 7.2 and 7.7.

Preferably, the culture DO is between 1% and 100%, more preferably between 10% and 100%.

Advantageously, the perfusion bioreactor culture step makes it possible to concentrate the culture cells to levels unattainable in batch and fed-batch culture, i.e. beyond 30 million cells/ml and up to 200 million cells/ml. Advantageously also, the perfusion bioreactor culture step of the method of the invention can make it possible to carry out differentiation of the cultured cells. Advantageously, in the case of the production of cultured red blood cells, the rate of enucleated cells at the end of the culture of the perfusion bioreactor culture step exceeds 50%, 60%, 70% or 80%.

In one embodiment of the invention, the perfusion culture step according to the invention is preceded by at least one culture step in a batch or fed-batch bioreactor.

In batch cultures, the medium is not renewed, so the cells only have a limited quantity of nutrients available. Fed-batch culture corresponds to batch culture with feeding of nutrients, in particular, and/or culture medium.

The purpose of the batch or fed-batch bioreactor culture step(s) is to pre-amplify the cells to be cultured and, in the case of the production of cultured red blood cells, to engage or differentiate the starting cells, or to reinforce their engagement or differentiation, in the erythroid lineage.

Thus, in the case of the production of cultured red blood cells, it is possible, in one embodiment of the invention, to continue the culture step in a batch or fed-batch type bioreactor until the cultured cells are engaged in the erythroid lineage. According to this embodiment of the invention, cells are considered to be sufficiently engaged in the erythroid lineage when they exhibit one or more specific characteristics of the erythroid lineage, such as a percentage of cells exhibiting the CD235 marker, measurable for example by flow cytometry, greater than 50%, or a percentage of cells with an erythroid phenotype, measurable for example by cytological counting after staining with May-Grunwald Giemsa dye, greater than 50%.

One or more successive or iterative cultures in a batch or fed-batch bioreactor can be carried out, for example between 1 and 4 times.

The batch or fed-batch bioreactor model is not particularly limited as long as it can generally culture animal cells. Preferably, the batch or fed-batch type bioreactor has a capacity of 0.5 to 5000 L, more preferably 0.5 to 500 L.

In one embodiment of the invention, the method for producing culture cells according to the invention comprises a step of purifying the culture cells obtained after the step of culture in a perfusion bioreactor.

The purification step aims to:
wash the cells to remove potentially toxic residues from the process; and
in the case of the production of cultured red blood cells, to sort the cells so as to concentrate the enucleated cells as much as possible.

The purification step may include one or more operations, including a particle sorting operation and a washing operation. The washing operation can be carried out either before or after the particle sorting operation.

In the case of the production of cultured red blood cells, particle sorting makes it possible to increase the rate of enucleated cells, in particular by eliminating erythroblasts and any residual myeloid cells. Erythroblasts are cultured cells that have not reached the stage of differentiation into enucleated cells, that is, into reticulocytes or red blood cells. Particle sorting also allows the removal of cellular waste, such as cellular debris, DNA and pyrenocytes.

The particle sorting according to the invention may comprise at least one operation selected from the group consisting of tangential filtration, frontal filtration and elutriation.

Tangential-flow filtration is well known to those skilled in the art. It is a filtration process that separates particles from a liquid based on their size. In tangential filtration, the liquid flow is parallel to the filter, unlike frontal filtration (or “dead-end filtration”) in which the liquid flow is perpendicular to the filter. It is the pressure of the fluid that allows it to pass through the filter. This results in the particles that are small enough pass through the filter while those that are too large continue on their way via the liquid flow.

Frontal filtration is well known to those skilled in the art. Its principle consists of retaining the particles to be eliminated inside a porous network constituting the filter. Filtration is based on 4 mechanisms: (i) particle/wall adhesion forces, (ii) inter-particle adhesion forces, (iii) steric hindrance and (iv) fluid drag force on particles. Its efficiency depends in particular on the material, the pore sizes, the type of fibre entanglement and the ratio of filtration surface to quantity of material to be filtered.

Elutriation is a technique for separation and granulometric analysis of particles of different sizes. Elutriation is based on Stokes' law. A fluid containing the cells is sent into a chamber at a known speed where the particles are subjected to a controlled centrifugal force. The latter remain in suspension when the two forces (fluid drive and centrifugal) cancel each other out.

Preferably, the particle sorting operation according to the invention comprises a succession of frontal filtrations and possibly elutriation.

The washing operation is intended in particular to reduce the quantities of toxic compounds potentially present in the culture of cells in a perfusion bioreactor below their toxicity threshold.

The washing operation may include one or more centrifugation operations and/or one or more elutriation operations.

Centrifugation is well known to those skilled in the art. It is a process of separating compounds from a mixture based on their density difference and drag by subjecting them to a unidirectional centrifugal force and possibly an opposing flow.

Preferably, the washing step according to the invention comprises a succession of elutriation operations.

The particle sorting, washing and formulation steps are carried out in a time period of less than 72 hours, more preferably less than 12 hours.

### Culture Medium

The person skilled in the art is able to select or prepare a suitable culture medium according to the invention. Examples of suitable culture media include those described in international publication WO2011/101468A1 and in the article Giarratana et al. (2011) “Proof of principle for transfusion of in vitro-generated red blood cells”, Blood 118:5071-5079.

The culture medium generally comprises a basal culture medium for eukaryotic cells, such as DMEM, IMDM, RPMI 1640, MEM or DMEM/F12 medium, which are well known to those skilled in the art and widely commercially available.

The culture medium or perfusion fluid may also include plasma, particularly in an amount of 0.5% to 6% (v/v).

Preferably, the culture medium or perfusion fluid further comprises nutrients and growth factors, cytokines and/or hormones.

Thus, the person skilled in the art is able to adapt the culture medium and the perfusion liquid by adding certain components or by modulating the quantities of certain components, in particular sodium, potassium, calcium, magnesium, phosphorus, chlorine, various amino acids, various nucleosides, various vitamins, various antioxidants, fatty acids, sugars and the like, foetal bovine serum, human plasma, human serum, horse serum, heparin, cholesterol, ethanolamine, sodium selenite, monothioglycerol, mercaptoethanol, bovine serum albumin, human serum albumin, sodium pyruvate, polyethylene glycol, poloxamers, surfactants, lipid droplets, antibiotics, agar, collagen, methylcellulose, various cytokines, various hormones, various growth factors, various small molecules, various extracellular matrices and various cell adhesion molecules.

Examples of cytokines included in the culture medium or perfusion fluid include interleukin-1 (IL-1), interleukin-2 (IL-2), interleukin-3 (IL-3), interleukin-4 (IL-4), interleukin-5 (IL-5), interleukin-6 (IL-6), interleukin-7 (IL-7), interleukin-8 (IL-8), interleukin-9 (IL-9), interleukin-10 (IL-10), interleukin-11 (IL-11), interleukin-12 (IL-12), interleukin-13 (IL-13), interleukin-14 (IL-14), interleukin-15 (IL-15), interleukin-18 (IL-18), interleukin-21 (IL-21), Interferon-A (IFN-α), interferon-β (IFN-β), interferon-γ (IFN-γ), granulocyte colony-stimulating factor (G-CSF), monocyte colony-stimulating factor (M-CSF), granulomacrophage cell colony-stimulating factor (GM-CSF), stem cell factor (SCF), flk2/flt3 ligand (FL), leukemia cell inhibitory factor (LIF), oncostatin M (OM), erythropoietin (EPO), thrombopoietin (TPO) However, it is not limited to the aforesaid.

Various small molecules included in the culture medium or perfusion fluid may include, but are not limited to, aryl hydrocarbon receptor antagonists such as StemRegeninl (SRI), hematopoietic stem cell self-renewal agonists such as UMI 71, and the like, but without being limited there to.

Growth factors included in the culture medium or perfusion fluid may include transforming growth factor-α (TGF-α), transforming growth factor-β (TGF-β), macrophage inflammatory protein-1α (MIP-1α), epidermal growth factor (EGF), fibroblast growth factor-1, 2, 3, 4, 5, 6, 7, 8 or 9 (FGF-1, 2, 3, 4, 5, 6, 7, 8, 9), nerve cell growth factor (NGF), vasculo-endothelial growth factor (VEGF), hepatocyte growth factor (HGF), leukemia inhibitory factor (LIF), nexin I protease, nexin II protease, platelet-derived growth factor (PDGF), cholinergic differentiation factor (GDF), various chemokines, Notch ligands (such as Delta 1), Proteins Wnt, angiopoietin-like proteins 2, 3, 5 or 7 (Angpt 2, 3, 5, 7), insulin-like growth factors (GF), insulin-like growth factor binding protein (IGFBP), pleiotrophin, and the like, but without being limited there to.

Hormones included in the culture medium or perfusion fluid may include hormones, in particular, from the family of glucocorticoids such as dexamethasone or hydrocortisone, from the family of thyroid hormones such as T3 and T4, ACTH, alpha-MSH, or insulin.

Preferably, particularly in the case of production of cultured red blood cells, the bioreactor is supplied, particularly via the perfusion liquid, with a source of ferric iron. More preferably, the source of ferric iron is a complex of ferric iron and a chelating agent, especially the citrate.

Preferably, the culture medium comprises transferrin, in particular, recombinant transferrin. Preferably, the transferrin concentration in the bioreactor is 10 to 3,000 μg/ml, more preferably 10 to 500 μg/ml.

### Antioxidant Capacity

The person skilled in the art is familiar with the methods for determining the antioxidant capacity of a solution, in particular a culture medium, and comparing it to the antioxidant capacity of a Trolox solution. A method that can be used is for example described in the article Marc et al. (2004) Med Sci (Paris) 20:458-463. Alternatively, commercial tests are available, for example OxiSelect™ (Cell Biolabs, Inc).

As an example, the antioxidant capacity of a solution can be deduced from its capacity to inhibit the ABTS.+ radical, obtained from ABTS (ammonium salt of 2,2′-azinobis-(3-ethylbenzothiazoline-6-sulfonic acid)) compared to a solution of a reference antioxidant: Trolox (6-hydroxy-2,5,7,8-tetramethylchroman-2-carboxylic acid). The cation radical is obtained by contact of ABTS with a peroxidation enzyme, such as horseradish peroxidase, in the presence of H₂O₂ or an oxidant, such as manganese dioxide or potassium persulfate. The ABTS.+ radical, in contact with an H. donor, leads to ABTS+ and to the discoloration at 734 nm of the solution. Other authors use 2,2′-azinobis-(3-ethylbenzothiazoline-6-sulfonic acid), or ABTS.−, instead of its ammonium salt and analyse the inhibition of the radical ABTS.−, produced by an initiator of thermolabile radicals, ABAP (2,2′-azobis-(2-amidinopropane)HCl). The reaction kinetics of the antioxidant solution studied must be examined beforehand to determine the end of the reaction.

As the person skilled in the art will understand, the antioxidant capacity of a volume of culture medium is compared to the antioxidant capacity of the same volume of Trolox solution.

Preferably, the antioxidant capacity of the culture medium is greater than or equal to the antioxidant capacity of a Trolox solution at 10 μM, 20 μM, 30 μM, 40 μM, 50 μM, 60 μM, 70 μM, 75 μM, 80 μM, 90 μM, 100 μM, 110 μM, 120 μM, 130 μM, 140 μM, 150 μM, 160 μM, 170 μM, 180 μM, 190 μM, 200 μM, 210 μM, 220 μM, 230 μM, 240 μM or 250 μM.

Preferably, the antioxidant capacity of the culture medium is less than or equal to the antioxidant capacity of a Trolox solution at 1000 μM, 750 μM, 500 μM, 400 μM, 300 μM, 250 μM, 200 μM, 150 μM or 100 μM.

Preferably, the antioxidant capacity of the culture medium is equal to the antioxidant capacity of a Trolox solution of 10 μM to 500 μM, 10 μM to 250 μM, from more than 50 μM to less than 100 μM, in particular from 51 μM to 99 μM, or from 75 μM to 150 μM.

### Antioxidant Agent

The antioxidant agent is preferably water-soluble.

Preferably, the antioxidant agent is selected from the group consisting of a water-soluble analogue or derivative of vitamin E, a carotenoid, especially beta-carotene or lycopene, ascorbic acid, selenium, glutathione (GSH), β-mercaptoethanol, uric acid, uracil, N-acetyl-cysteine, tempol, NADPH, NADH, astaxanthin, lutein, zeaxanthin, retinol, retinal, retinoic acid, allicin, alliin, allyl cysteine, allyl disulfide, melatonin, nuphlutin, hermidine, resveratrol, catechin, beta-hydroxy acid (BHA), caffeic acid, curcumin, ferulic acid, 8-hydroxyl quinoline, isoferulic acid, maclurine, magnolol, MEAS (methanolic extract of Aquilaria sinensis leaves), MEGM (methanolic extract of Gynura bicolor Roxb. DG.), proanthocyanidin, protocatechuic acid, puerarin, pyridoxine, quercetin, rutin, and an antioxidant protein.

Preferably, the antioxidant protein is selected from the group consisting of a thiol protein, haptoglobin, hemoplexin, catalase, peroxidase, including ascorbate peroxidase, guaiacol peroxidase, or glutathione peroxidase, superoxide dismutase, peroxiredoxin, glutaredoxin, thioredoxin and glutathione reductase.

Preferably, the antioxidant agent is ascorbic acid or a water-soluble analogue or derivative of vitamin E.

As used herein, the water-soluble analogue or derivative of vitamin E is in particular a water-soluble analogue or derivative of tocopherol, in particular alpha-tocopherol. Preferably, the water-soluble analogue or derivative of vitamin E is Trolox, Tocofersolan, or MDL 73404, more preferably Trolox.

Trolox is also called 3,4-dihydro-6-hydroxy-2,5,7,8-tetramethyl-2H-I-benzopyran-2-carboxylic acid and is referenced under the CAS number 53188-07-1.

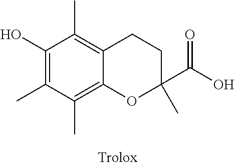 [SMILES: Cc1c(C)c2c(c(C)c1O)CCC(C)(C(=O)O)O2]

Ascorbic acid is also named 5-(1,2-dihydroxyethyl)-3,4-dihydroxyfuran-2-one. The ascorbic acid according to the invention may be L-ascorbic acid (vitamin C), D-ascorbic acid or a mixture of L-ascorbic acid and D-ascorbic acid.

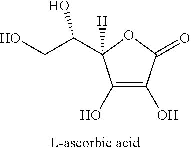 [SMILES: O=C1O[C@H]([C@@H](O)CO)C(O)=C1O]

Preferably, the culture medium comprises ascorbic acid or Trolox at a concentration of at least 10 μM, 20 μM, 30 μM, 40 μM, 50 μM, 60 μM, 70 μM, 75 μM, 80 μM, 90 μM, 100 μM, 110 μM, 120 μM, 130 μM, 140 μM, 150 μM, 160 μM, 170 μM, 180 μM, 190 μM, 200 μM, 210 μM, 220 μM, 230 μM, 240 μM or 250 μM.

Preferably, the culture medium comprises ascorbic acid or Trolox at a concentration of at most 1000 μM, 750 μM, 500 μM, 400 μM, 300 μM, 250 μM, 200 μM, 150 μM or 100 μM.

Preferably, the culture medium comprises ascorbic acid or Trolox at a concentration of 10 μM to 500 μM, 10 μM to 250 μM, more than 50 μM to less than 100 μM, especially 51 μM to 99 μM, or 75 μM to 150 μM.

### Cells

The cells according to the invention are of any type requiring a supply of ferric iron.

Preferably, they are eukaryotic cells, more preferably animal cells, in particular bird, mammal or human cells.

These may be cells to be cultured for their own sake, such as NK cells, lymphocytes, including chimeric antigen receptor T cells (CAR T cells), erythroid cells, including erythroblasts, cultured red blood cells or cultured meat cells, or cells to be cultured in order to produce molecules of interest, in particular proteins, more particularly antibodies or antibody derivatives, in particular monoclonal antibodies. Preferably, the cells to be cultured are the site of heme production.

Preferably, the culture cells requiring ferric iron supply are cells that contain hemoglobin and/or myoglobin.

Preferably, the cultured cells requiring ferric iron supply are erythroid cells, including erythroblasts, cultured red blood cells or cultured meat cells. Preferably, the cultured cells are cultured erythroblasts or red blood cells.

As we understand it here, “cultured meat” is synonymous with “synthetic meat” or even “clean meat”.

The cells according to the invention may be stem cells, progenitors, or cells of an immortalised cell line of the erythroid lineage.

Stem cells can be embryonic stem cells (ESCs), induced pluripotent stem cells (iPSCs), or hematopoietic stem and/or progenitor cells (HSCs/HPs). Preferably, the method according to the invention uses as cell source hematopoietic stem cells and/or hematopoietic progenitors (HSC/HP).

Cells of an immortalised cell line of the erythroid lineage can be immortalised at the stage of an erythroid progenitor or an erythroid precursor. Furthermore, hematopoietic stem cells (HSCs) can also be immortalised.

Immortalisation is preferably carried out conditionally. These immortalised cells can then be passaged indefinitely in vitro, cryopreserved and recovered, and, conditionally, produce fully differentiated red blood cells from a defined and well-characterised source. Conditional immortalisation can be achieved by any method well known to those skilled in the art.

Embryonic stem cells (ESCs) and induced pluripotent stem cells (iPSCs) are pluripotent stem cells. These cells are both capable of differentiation into many cell types and capable of self-replication. They can maintain this pluripotency of differentiation while multiplying by division. Embryonic stem cells refer to pluripotent stem cells derived from embryos at the blastocyst stage, which is the early stage of animal development. Induced pluripotent stem cells (iPSCs) are produced by introducing several types of transcription factor genes into somatic cells such as fibroblasts.

The embryonic stem cells (ESC) according to the invention are obtained by any means not requiring the destruction of human embryos. For example, using the technology described by Chung et al. (Chung et al, Human Embryonlc Stem Cell Unes generated without embryo destruction, Cell Stem Cell (2008)). Furthermore, the method according to the invention does not use human embryos in any case and does not aim in any case to induce the development process of a human being.

According to one embodiment of the invention, the said stem cells used in the method according to the invention are not human embryonic stem cells (hESC) and/or iPSC.

The hematopoietic stem cells (HSCs) used in the method according to the invention are multipotent cells. They are capable of differentiating into all blood cell differentiation lineages and capable of self-replication while maintaining their multipotency.

Cells of an immortalised cell line of the erythroid lineage are cells already committed to the erythroid lineage but capable of self-replication and under external control of differentiating into cells of the erythroid lineage.

The hematopoietic stem and/or progenitor cells (HSC/HP) used in the method according to the invention may be derived from any source, including, being derived from bone marrow, umbilical cord/placental blood or peripheral blood with or without prior mobilisation.

The origin of stem cells and cells of an immortalised cell line of the erythroid lineage is not particularly limited as long as it is derived from a mammal. Preferred examples include humans, dogs, cats, mice, rats, rabbits, pigs, cows, horses, sheep, goats and the like, with humans being most preferred.

The cells used in the method of the invention may produce, without limitation, universal donor red blood cells, rare blood group red blood cells, red blood cells for personalised medicine (e.g., autologous transfusion, optionally with genetic engineering), and red blood cells designed to include one or more proteins of interest.

In certain embodiments which may be combined with any of the preceding embodiments, the said cells used in the method according to the invention may be isolated from a patient having a rare blood group including, without limitation, Oh, ODE/ODE, CdE/CdE, CwD−/CwD−, −D−/−D−, Rhnull, Rh: −51, LW (a−b+), LW (ab−), SsU−, SsU (+), pp, Pk, Lu (a+b−), Lu (ab−), Kp (a+b−), Kp (ab−), Js (a+b−), Ko, K: −11, Fy (ab−), Jk (ab−), Di (b−), I−, Yt (a−), Sc: −1, Co (a−), Co (ab−), Do (a−), Vel−, Ge−, Lan−, Lan (+), Gy (a−), Hy−, At (a−), Jr (a−), In (b−), Te (a−), Cr (a−), Er (a−), Ok (a−), JMH− and En (a−).

According to one embodiment of the invention, the said cells may be embryonic stem cells (ESC), preferably human (hESC) and preferably selected from the group consisting of the lines H1, H9, HUES-1, HUES-2, HUES-3, HUES-7, CLO1 and pluripotent stem cells (iPSC), preferably human (hiPSC)

Preferably, the said cells are hematopoietic stem cells and/or hematopoietic progenitors (HSC/HP), more preferably human.

In the case of cells derived from umbilical cord/placental blood or peripheral blood, bone marrow, or from apheresis sample, a specific cell selection step for CD34+ cells may be carried out before the batch or fed-batch bioreactor culture step of the process according to the invention.

Apheresis is a technique of collecting certain blood components by extra-corporeal circulation of blood. The components that are to be collected are separated by centrifugation and extracted, while the components not collected are re-injected into the donor (blood) or the patient (therapeutic apheresis).

The term CD34+ (positive) means that the CD (cluster of differentiation) 34 antigen is expressed on the surface of the cells. This antigen is a marker of hematopoietic stem cells and hematopoietic progenitor cells, and disappears as they differentiate themselves. Similar cell populations also include CD133-positive cells.

In the case where the original cells are ESCs, iPSCs or cells of an immortalised cell line of the erythroid lineage, pre-culture steps can be added upstream of the culture step in the bioreactor to multiply the cells and possibly engage them in a differentiation pathway, in particular of the erythroid lineage.

Preferably, the culture cells requiring ferric iron supply are cultured red blood cells and the cells to be cultured are erythroid stem cells or progenitors or cells of an immortalised cell line of the erythroid lineage.

Regardless of the cell source, a prior step of freezing the cells to be cultured is often required for transport and conservation reasons. Cell freezing methods are well known in the state of the art and notably involve a programmed temperature reduction as well as the use of cryoprotectants such as lactose or dimethylsulfoxide (DMSO). When added to the medium, DMSO prevents the formation of intracellular and extracellular crystals in cells during the freezing process.

Thus, in a particular embodiment of the invention, the method according to the invention comprises a step of thawing the cells, prior to the step of culture in a perfusion bioreactor, in the case where the cells to be cultured are frozen. Methods of thawing cells are well known to those skilled in the art.

Thawing is a step in the process that should not be neglected, especially when DMSO has been used for freezing. This compound is indeed cryo-preservative as long as the cell suspension is stored in liquid nitrogen or nitrogen vapour. However, it becomes cytotoxic as soon as the cell suspension is thawed. It is therefore appropriate to remove the DMSO very quickly by several washing steps as soon as the cells have thawed, as is well known to those skilled in the art.

In other cases, the starting cells can be fresh, that is to say the time between cell collection and culturing is short enough not to require freezing, preferably this time is less than 48 hours. This situation may exist, for example, when the sampling centre is located on the same site or near the production centre.

The invention will be further explained with the aid of the following non-limiting Example and Figures.

### DESCRIPTION OF FIGURES

FIG. 1 shows cell losses at the end of culture (%, y-axis) during red blood cell perfusion cultures conducted in the presence (yes) or absence (no) of Trolox in the culture medium.

FIG. 2 shows the red blood cell preservation yield (%, y-axis) of red blood cells obtained by perfusion cultures conducted in the presence (yes) or absence (no) of Trolox in the culture medium.

### EXAMPLE

Cultured red blood cell production was performed with and without the addition of Trolox during the perfusion bioreactor culture step of the process described below.

Briefly, the cells cultured according to the invention are total nucleated cells collected by cytapheresis from volunteer donors previously mobilised with G-CSF.

A first step of the process according to the invention is carried out over 7 days (from D1 to D7) in fed-batch at a temperature of 37° C., under an atmosphere of 5% CO2 and in a culture medium adapted from that described by Giarratana et al. (2011) “Proof of principle for transfusion of in vitro-generated red blood cells”, Blood 118:5071-5079 for the first step of the expansion procedure described in the article (page 5072). Halfway through this step, fresh culture medium is added to the culture so as to dilute the culture by half (the same volume of culture medium is added as the volume initially present).

A second step of the process according to the invention is carried out over 15 days (D7 to D22) in a 2 L perfusion bioreactor equipped with a tangential filtration system and a centrifugal (TFF) or diaphragm (ATF) pump. The culture is carried out at a temperature of 37° C., under an atmosphere of 5% CO2, with a culture medium similar to that of the previous step except that IL-3 and glucocorticoid are absent. Occasional contributions of SCF and EPO are also made as well as a continuous iron intake.

The second step is carried out in the absence or presence of Trolox added to the culture medium at a concentration of approximately 100 μM.

Fifteen cultures were performed (9 without Trolox and 6 with Trolox) and cell losses were measured at the end of the culture, defined as the % decrease in cell concentration between the peak cell concentration and the day the culture was stopped (2 to 3 days later).

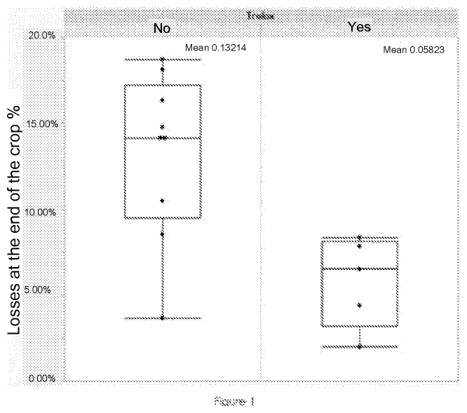

FIG. 1 shows that the addition of Trolox to the culture medium significantly reduces cell losses at the end of culture (13% loss without Trolox versus 6% loss with Trolox on average).

Furthermore, the red blood cells from the above cultures were stored for 28 days in a preservation solution and the preservation efficiency at the end of the preservation period was determined [100×(final red blood cell count/mL)/(initial red blood cell count/mL].

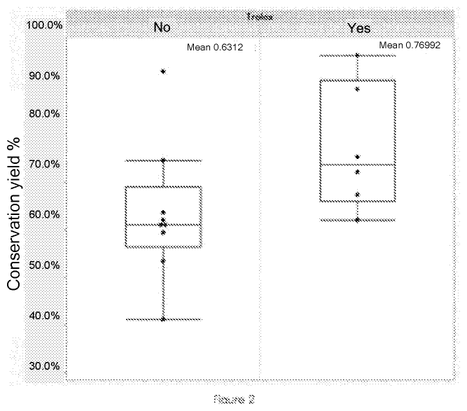

FIG. 2 shows that the average conservation yield is significantly improved when the culture was conducted in the presence of Trolox (77% in the presence of Trolox versus 63% without Trolox).

1. A method for culturing cells requiring a supply of iron, comprising a step of culturing cells to be cultured in a culture medium whose antioxidant capacity is greater than or equal to the antioxidant capacity of a 10 μM Trolox solution.

2. The method for culturing of claim 1, wherein the culture medium comprises at least one antioxidant agent.

3. The method for culturing of claim 2, wherein the antioxidant agent is ascorbic acid or a water-soluble analogue or derivative of vitamin E.

4. The method for culturing of claim 1, wherein the culture medium comprises ascorbic acid or Trolox at a concentration of at least 10 μM.

5. The method for culturing of claim 1, wherein the antioxidant capacity of the culture medium is greater than or equal to the antioxidant capacity of a 50 μM Trolox solution.

6. The method for culturing of claim 1, wherein the cells to be cultured are the site of heme production.

7. The method for culturing of claim 1, wherein the culture cells are erythroblasts or cultured red blood cells.

8. The method for culturing of claim 1, wherein the method for culturing is a perfusion culturing method.

9. A culturing medium for the culture of cells requiring a supply of iron, the antioxidant capacity of which is greater than or equal to the antioxidant capacity of a 10 μM Trolox solution, and is notably lower than the antioxidant capacity of a 250 μM Trolox solution.

10. The culturing medium according to claim 9, comprising at least one antioxidant agent.

11. The culturing medium according to claim 10, wherein the antioxidant agent is ascorbic acid or a water-soluble analogue or derivative of vitamin E.

12. The culturing medium according to claim 9, comprising ascorbic acid or Trolox at a concentration of at least 10 μM.

13. The method for culturing of claim 3, wherein the water-soluble analogue or derivative of vitamin E is Trolox or Tocofersolan.

14. The culturing medium according to claim 9, having an antioxidant capacity which is greater than or equal to the antioxidant capacity of a 50 μM Trolox solution.

15. The culturing medium according to claim 11, wherein the water-soluble analogue or derivative of vitamin E is Trolox, Tocofersolan, or MDL 73404.

16. The culturing medium according to claim 12, comprising ascorbic acid or Trolox at a concentration of at least 50 μM.
